(19) United States
(12) Patent Application Publication
(10) Pub. No.: US 2004/0044264 A1
(43) Pub. Date: Mar. 4, 2004

(54) Use of ionic liquids to separate olefins, diolefins and aromatics

(75) Inventors: Ronald Scott Smith, Calgary (CA); Patricio S. Herrera, Calgary (CA); Sean Reynolds, Calgary (CA)

Correspondence Address: Kenneth H. Johnson, Patent Attorney, P.O. Box 630708, Houston, TX, 77263, US

(73) Assignee: NOVA Chemicals (International) S.A.

(21) Appl. No.: 10/648,611

(22) Filed: Aug. 26, 2003

(30) Foreign Application Priority Data
Aug. 28, 2002 (CA) 2,400,714

(51) Int. Cl. C07C 7/10; C07C 7/148; C07C 7/00

(52) U.S. Cl. 585/862; 585/845; 585/843

(57) ABSTRACT

The present invention relates to the separation of diolefins, and lower aromatics from mixed streams of hydrocarbons using ionic liquids and metal complexes. The present invention provides a novel method to separate diolefins and lower aromatics from other hydrocarbyl streams which may contain small amounts of water.

### FIELD OF THE INVENTION

The present invention relates to the separation of diolefins and lower aromatic compounds from hydrocarbons using metal complexes in ionic liquid.

### BACKGROUND OF THE INVENTION

In a number of processes, including cracking of feedstocks, there is produced a stream comprising one or more diolefins together with other hydrocarbon compounds. The aromatic and diolefin components may be separated from the stream by distillation but this technique is energy intensive. It is desirable to find a lower energy method to separate diolefins and lower aromatics from hydrocarbyl streams.

It is known to separate olefins from paraffins by forming complexes with metals such as silver or copper. The resulting copper or silver complex is preferentially soluble in a liquid not miscible or soluble in the paraffin, such as water. The streams are separated and then the olefin is released from the complex typically by a temperature or pressure change. The regenerated metal compound is then capable of being reused to complex more olefin. In some cases the metal compound is adsorbed or complexed on the surface of an ion exchange resin or in a membrane separation film and the olefin is separated from the alkane. Representatives of such art include Canadian Patent 1,096,779 issued Mar. 3, 1981 to Deutsche Texaco A. G.; U.S. Pat. No. 3,979,280 issued Sep. 7, 1976 and assigned to Deutsche Texaco A. G.; U.S. Pat. No. 4,328,382 issued May 4,1982 assigned to Erdoelchemie G.m.b. H.; and U.S. Pat. No. 3,441,377, issued Apr. 29,1969 to Esso Research and Engineering Co. This art does not teach or suggest separation of diolefins and lower aromatics using a complexing technique.

Most recent in this line of technology is U.S. Pat. No. 6,339,182 B1 issued Jan. 15, 2002 to Munson et al., assigned to Chevron U.S.A. Inc. This patent teaches the absorption of alkenes by metal salts, typically silver or copper salts in ionic liquids. The alkenes are typically initially present as an admixture with paraffins. The alkenes are regenerated by separation from the metal complex by temperature or pressure change or application of an entrainment gas such as an inert gas. The reference does not teach or disclose the separation of lower aromatics or diolefins using comparable techniques.

Applicants have discovered that diolefins and lower aromatics form complexes with silver or copper salts which are preferentially soluble in some ionic liquids and which may be dissociated from the metal complex under fairly mild conditions.

The present invention seeks to provide a simple process for the separation of diolefins and lower aromatics from other hydrocarbons, particularly alkanes.

### SUMMARY OF THE INVENTION

The present invention provides a process for separating one or more members selected from the group consisting of C₄₋₈ diolefins and C₆₋₁₂ aromatic hydrocarbons which are unsubstituted or substituted by up to three C₁₋₄ alkyl radicals from a mixture comprising at least one of said members and at least one other hydrocarbon comprising contacting said mixture with a copper or silver complexing compound in a nitrogen containing ionic liquid having a melting temperature below. 80° C. to preferentially take said one or more members into said ionic liquid, separating said ionic liquid from said at least one other hydrocarbon and regenerating said ionic liquid and releasing said at least one member.

### DETAILED DESCRIPTION

In accordance with the present invention one or more members selected from the group consisting of C₄₋₈ conjugated diolefins, and C₆₋₈ aromatic hydrocarbons may be separated from one or more hydrocarbons, typically paraffins, typically having up to about 20 carbon atoms, preferably C₁₋₁₈ paraffins.

The diolefins are typically C₄₋₈ diolefins. The diolefins may be conjugated or non-conjugated. Some diolefins include butadiene, including 1,3-butadiene, hexadiene including 1,4-hexadiene and 1,5-hexadiene, and octadiene including 1,7-octadiene. The dienes may be substituted by a C₁₋₄ alkyl radical such as isoprene. Preferably the dienes are hydrocarbyl olefins and do not contain other atoms or functional groups.

The C₆₋₁₂ aromatic compounds are also preferably hydrocarbyl compounds. These compounds may be unsubstituted or may be substituted by up to three lower alkyl groups (i.e. C₁₋₄ alkyl radicals). This group of compounds includes benzene, toluene, xylene, and naphthalene.

Preferably the compounds being separated from the mixture with other hydrocarbons are mixtures containing one or more diolefins and/or one or more lower aromatic compounds.

The mixtures to be treated in accordance with the present invention may be subject to a number of treatments prior to being contacted with the ionic liquid. Such treatments are well known to those skilled in the art and include for example removal of polar species (e.g. CO, CO₂ and water) and hydrogenation such as hydrogenation of acetylenes.

The copper and silver complexes may be selected from the group consisting of silver acetate, silver nitrate, and silver tetrafluorborate and mixtures thereof. The copper and silver complexes may be present in the ionic liquid in an amount from about 5 to 50, preferably 5 to 25 most preferably 5 to 15 weight %.

Ionic liquids are organic compounds that are liquid at room temperature. They differ from most salts, in that they have very low melting points. They tend to be liquid over a wide temperature range and have essentially no vapor pressure. Most are air and water stable, and they are used herein to solubilize olefins, diolefins, and/or aromatic hydrocarbons. The properties of the ionic liquids can be tailored by varying the cation and anion. Examples of ionic liquids are described, for example, in J. Chem. Tech. Biotechnol., 68:351-356 (1997); Chem. Ind., 68:249-263 (1996); and J. Phys. Condensed Matter, 5 :(supp 34B):B99-B106 (1993), Chemical and Engineering News, Mar. 30, 1998, 32-37; J. Mater. Chem., 8:2627-2636 (1998); and Chem. Rev., 99:2071-2084 (1999), the contents of which are hereby incorporated by reference.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various counter ions such as Lewis acids or their conjugate bases to form ionic liquids (nitrogen based ionic liquid). Examples of suitable heterocyclic rings include substituted pyridines, imidazole, substituted imidazole, pyrrole and substituted pyrroles. These rings can be alkylated with virtually any straight, branched or cyclic C₁₋₂₀ alkyl group, but preferably, the alkyl groups are C₁₋₆ groups, since groups larger than this tend to increase the melting point of the salt.

Ionic liquids have also been based upon various triarylphosphines, thioethers, and cyclic and non-cyclic quaternary ammonium salts. Counterions which have been used include chloroaluminates, bromoaluminates, gallium chloride, tetrafluoroborate, tetrachloroborate, hexafluorophosphate, nitrate, trifluoromethane sulfonate, methylsulfonate, p-toluenesulfonate, hexa fluoroantimonate, hexa fluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions.

In accordance with the present invention the organic portion of the ionic liquid is typically a nitrogen containing C₅₋₈ hetrocyclic aromatic compound. The heterocyclic aromatic compound may be unsubstituted or substituted by up to three C₁₋₆ alkyl radicals. The heterocyclic aromatic compound may be selected from the group comprising pyrrolium, imidazolium, and pyridinium which are unsubstituted or substituted by up to two C₁₋₄ alkyl radicals, for example 1-butyl-3-methylimidazolium and 4-methyl-N-butylpyridinium.

Useful counter ions include borate compounds, preferably tetrahaloborates most preferably tetrafluoroborate (the corresponding acid form of Lewis acid would for example be H⁺BF⁻₄). Other counter-ions which may be suitable for use in the present invention are discussed in U.S. Pat. No. 6,339,182.

Some ionic liquids which may be used in accordance with the present invention include 1-butyl-3-methylimidazolium tetrafluoroborate; 1-hexyl-3-methylimidazolium tetrafluoroborate; 4-methyl-N-butylpyridinium tetrafluoroborate; 4-hexyl-N-butylpyridinium tetrafluoroborate; N-butylpyridinium tetrafluoroborate and N-hexylpyridinium tetrafluoroborate. Further or differently substituted homologues of these compounds are within the scope of the present invention. Other ionic liquids would be apparent to those skilled in the art.

The ionic liquid may optionally contain from 0 up to about 15, preferably less than 10% by volume of water.

The above noted diolefins and aromatic hydrocarbons can be selectively separated from mixtures containing one or more of such compounds and other hydrocarbons such as paraffins and higher aromatics. The separation involves contacting the mixture containing one or more of the diolefins and lower aromatic hydrocarbons with one or more of the silver or copper complexes in an ionic liquid. The silver or copper complex takes up (further complexes) the diolefins and lower aromatic compounds present in the mixture. The ionic liquid is then separated from the hydrocarbyl mixture (which has a significantly (e.g. 75%) reduced content of such diolefins and lower aromatic compounds. The hydrocarbon stream can be separated from the ionic liquid using conventional means including, for example, decantation, and the like. In the separation of the residual hydrocarbon stream from the ionic liquid care needs to be taken not to subject the ionic liquid to conditions which will cause it to give up the one or more of the olefins, diolefins, and lower aromatic compounds.

The mixture containing one or more of the diolefins and lower aromatic hydrocarbons may be contacted with the ionic liquid containing one or more copper or silver complexes using well known methods including, co-current, counter-current, or staged in stirred tanks. Countercurrent methods are preferred. The mixture containing one or more olefins, diolefins, or lower aromatic compounds can be in the gas phase or the liquid phase. The ionic liquid will be in the liquid phase. Typically the contact will take place at temperatures less than about 80° C., preferably less than 50° C. desirably less than 35° C., preferably about room temperature (i.e. from 15° C. to 25° C). The pressure may be low (i.e. up to 1000 psig (6,895 kPa), preferably less than 100 psig (689.5 kPa). If the contact with the ionic liquid is under pressure the pressure on the ionic liquid should not be reduced until it is desired to release the one or more olefins, diolefins and lower aromatic compounds from the ionic liquid.

The one or more of the diolefins and lower aromatic compounds may then be recovered from the copper and/or silver complex containing ionic liquids using a number of regeneration techniques. These techniques may include thermal regeneration (increasing the solution temperature to release the olefins, diolefins, and lower aromatic compounds); pressure swing regeneration (reducing the pressure) and combinations thereof. Entrainment gasses, typically inert gasses, preferably nitrogen may also be passed through the ionic liquid to entrain and release the olefins, diolefins, and lower aromatic hydrocarbons from the ionic liquid. Entrainment gasses may be used with either or both of the foregoing techniques to release the olefins, diolefins, and aromatic hydrocarbons from the ionic liquid.

Release of the one or more diolefins and lower aromatic compounds may be carried out in a packed tower or flash drum, preferably a packed tower generally by using a combination of increased temperature and/or lower pressure. The temperatures may range from about 100° C. to about 150° C. (although higher temperatures may be required for relatively high molecular weight diolefins, and aromatic compounds), preferably from about 120° C. to about 140° C., and the pressure may range from vacuum pressures to about 50 psig (345 kPa), preferably from about 10 psig (about 68.9 kPa) to about 30 psig (about 207 kPa). The temperatures should be higher, and the pressures should be lower for higher molecular weight diolefins and aromatic compounds. The decomposition temperature of the ionic liquids should not be exceeded.

The packed tower or flash drum may include multi-stage stripping or flashing for increased energy efficiency. In such systems, the ionic solution rich in a copper and/or silver complex of one or more diolefins and/or lower aromatic compounds is flashed and stripped at progressively higher temperatures and/or lower pressures. The design of such systems is well known to those skilled in the art.

Conventional heating means known to those of ordinary skill in the art, including steam and preferably low pressure steam, may be used to dissociate the one or more diolefins and lower aromatic compounds from the copper and/or silver complexes. One inexpensive heat source in the lower end of the temperature range is quench water. The packed column or flash drum is preferably equipped with a water wash section in the top to prevent entrainment of the desorbed gases.

The ionic liquid solution containing the copper or silver complex or both can then be removed from the bottom of the packed column or tower or flash drum and recycled back to the contact device.

The present invention provides a simple and relatively cheap means to separate butadiene and/or benzene from ethane in a flexi-cracker. Additionally, the present invention may be applied at the downsteam or back end of a solution or slurry polymerization and in particular a process which may be using dilute monomer as described in U.S. Pat. No. 5,981,818 issued Nov. 9, 1999 to Purvis et al., assigned to Stone & Webster Engineering Corp.

The present invention will now be illustrated by the following non-limiting examples in which unless otherwise indicated weight is in grams and parts is parts by volume.

### EXAMPLES

C₅ Hydrocarbon Solubility by Ionic Liquids

The present example investigated the solubility of diolefin C₅ hydrocarbons, isopentane and isoprene, in 1-butyl-3-methylimidazolium tetrafluoroborate (bmim⁺BF₄⁻) containing 1.69 mol/L of AgBF₄, and demonstrates the corresponding selectivity for diolefinic C₅'s over corresponding paraffin. The testing apparatus consisted of a flat-bottomed florence flask with a graduated neck. The flask was charged with 75 mL of bmim⁺BF₄⁻, the level being recorded. A known quantity of C₅ hydrocarbon was then added to the flask and the flask was sealed. The overall liquid level and the location of the liquid-liquid interfacial meniscus were recorded. The mixture was then agitated to contact the two liquids and the two phases were allowed to separate. The locations of all meniscuses were then recorded. Agitation and phase separation was then repeated until the liquid levels remained unchanged. The volume change of the hydrocarbon phase corresponds to the quantity of hydrocarbon dissolved in the ionic liquid. The testing was conducted at ambient temperature. The results are summarized in Table 1.

TABLE 1
C₅ Solubility in bmim⁺ BF₄⁻ Ionic Liquid Containing 1.69 M AgBF₄

| | Solubility | Solubility |
|---|---|---|
| Hydrocarbon | (mol C₅/L ionic liquid) | (mol C₅/mol Ag⁺) |
| Isopentane | 0.044 | 0.026 |
| Isoprene | 1.132 | 0.670 |

Cyclo-C₈ Hydrocarbon Solubility by Ionic Liquids

The procedure described in Example 1 was performed instead with ethylcyclohexane, ethylbenzene, and styrene. The ionic liquid used was 1-butyl-3-methylimidazolium tetrafluoroborate. Table 2 summarizes the findings.

TABLE 2
Cyclo-C₈ Solubility in bmim^{+ BF}₄⁻ Ionic Liquid Containing 1.69 M AgBF₄

| | Solubility | Solubility |
|---|---|---|
| Hydrocarbon | (mol C₈/L ionic liquid) | (mol C₈/mol Ag⁺) |
| Ethylcyclohexane | 0.10 | 0.06 |
| Ethylbenzene | 7.55 | 4.47 |
| Styrene | 7.01* | 4.15* |

*Solubility limit not reached due to experimental design limitations.

What is claimed is:

1. A process for separating one or more members selected from the group consisting of C₄₋₈ diolefins and C₆₋₁₂ aromatic hydrocarbons which are unsubstituted or substituted by up to three C₁₋₈ alkyl radicals from a mixture comprising at least one of said members and at least one other hydrocarbon comprising contacting said mixture with a copper or silver complexing compound in a nitrogen containing ionic liquid having a melting temperature below 80° C. to preferentially take said one or more members into said ionic liquid, separating said ionic liquid from said at least one other hydrocarbon and regenerating said ionic liquid and releasing said at least one member.

2. The process according to claim 1, wherein the copper or silver complexing compound is selected from the group consisting of silver acetate, silver nitrate, and silver tetrafluorborate and mixtures thereof.

3. The process according to claim 2, wherein in said ionic liquid the organic component is a heterocyclic nitrogen-containing aromatic compound.

4. The process according to claim 3, wherein said heterocyclic nitrogen containing aromatic compound is a C₅₋₈ nitrogen containing aromatic compound which is unsubstituted or substituted by up to three C₁₋₈ alkyl radicals.

5. The process according to claim 2, wherein said nitrogen-based ionic liquid is a nitrogen containing tetrafluoroborate ionic liquid.

6. The process according to claim 5, wherein said ionic liquid is selected from the group consisting of imidazolium and pyridinium ionic liquids which are unsubstituted or substituted by up to two C₁₋₈ alkyl radicals.

7. The process according to claim 6, wherein said ionic liquid is selected from the group consisting of 1-butyl-3-methylimidazolium tetrafluoroborate and 4-methyl-N-butylpyridinium tetrafluoroborate.

8. The process according to claim 7, wherein said mixture is in the gas or liquid state.

9. The process according to claim 8, wherein said regeneration of ionic liquid and said releasing of at least one member is effected using one or more treatments selected from the group consisting of increasing temperature, decreasing pressure, and passing an entraining gas through said ionic liquid.

10. The process according to claim 9, wherein said mixture and said ionic liquid are contacted in a counter-current flow.

11. The process according to claim 9, wherein said mixture and said ionic liquid are contacted in co-current flow.

12. The process according to claim 9, wherein said mixture and said ionic liquid are contacted in a continuous stirred tank reactor.

13. The process according to claim 1, wherein in the ionic liquid optionally contains from 0 to 15 volume % of water.

14. The process according to claim 6, wherein in the ionic liquid optionally contains from 0 to 15 volume % of water.

15. The process according to claim 7, wherein in the ionic liquid optionally contains from 0 to 15 volume % of water.
